(19) United States
(12) Patent Application Publication
(10) Pub. No.: US 2012/0295982 A1
(43) Pub. Date: Nov. 22, 2012

(54) TREATING HUMAN MALE COPD PATIENTS WITH ORAL BEDORADRINE

(75) Inventor: Kirk W. Johnson, Moraga, CA (US)

(73) Assignee: MediciNova, Inc.

(21) Appl. No.: 13/401,622

(22) Filed: Feb. 21, 2012

Related U.S. Application Data
(60) Provisional application No. 61/445,445, filed on Feb. 22, 2011.

(51) Int. Cl. A61K 31/165; A61P 11/00

(52) U.S. Cl. 514/620

(57) ABSTRACT

A method of treating a human male patient is disclosed. Enterally administering bedoradrine or a pharmaceutically acceptable salt thereof to a human male patient who is suffering from a medical condition that is responsive to bedoradrine or a pharmaceutically acceptable salt thereof, is useful and benefits human males versus human females. A preferred mode of administration is oral administration.

### CROSS-REFERENCE TO RELATED PATENT APPLICATIONS

This application claims priority from U.S. Provisional Application No. 61/445,445 filed on Feb. 22, 2011, which is incorporated herein by reference in its entirety.

### FIELD OF THE INVENTION

This invention relates to treating disorders of airway constriction or obstructive airways by administering bedoradrine (MN-221) enterally, preferably orally, particularly to male patients.

### BACKGROUND OF THE INVENTION

Chronic obstructive pulmonary disease or COPD refers to chronic bronchitis and/or emphysema, a pair of commonly co-existing diseases of the lungs in which the airways become narrowed. This condition leads to a limitation of the flow of air to and from the lungs causing shortness of breath. Symptoms of COPD include those that are consequences of the anatomical changes caused by the disease: barrel chest, pursed-lip breathing, productive cough, and cyanosis.

One telling symptom is the change in the shape of the chest, known as barrel chest. When the lungs become enlarged, the diaphragm is displaced downward and is unable to contract efficiently. Furthermore, the chest wall is enlarged, making accessory breathing muscles (muscles in the neck, upper chest, and between the ribs) less efficient as well. These changes contribute to shortness of breath.

This becomes apparent when a person with COPD tries do something with the arms raised above the head, such as changing a light bulb in a ceiling fixture, and becomes short of breath. To compensate, a person with COPD often sits leaning forward with their arms supported on a surface in front of them or on their knees. This stabilizes the upper chest and shoulders and allows them to use accessory respiratory muscles more efficiently. Because airflow out of the lungs becomes limited, exhalation takes longer. Because the alveoli lose their elasticity, one tries to shorten the time needed for exhalation by forcefully exhaling.

Unfortunately, forced exhalation increases pressure on the lungs and causes structurally weakened airways to collapse. To prevent airways from closing during forced exhalation, pursed-lip breathing is used: The lips are narrowed together, which slows exhalation at the mouth. This keeps positive pressure in the airways, thus preventing their collapse and allowing some forced exhalation. Furthermore, a productive cough is caused by inflammation and excessive amounts of mucus in the airways. Coughing becomes less effective because of obstructed airflow.

People who have a poor supply of oxygen usually have a bluish tinge to their skin, lips, and nailbeds, called cyanosis. Other symptoms of COPD, include dyspnea, chronic cough, and wheezing. Dyspnea, the most common symptom of COPD, comes on gradually and is first noticed during physical exertion or during acute exacerbations. It usually begins when patients are in their 60s and 70s and slowly becomes more prominent. It is closely associated with lung function decline and is not always associated with low oxygen in the blood. Patients often wonder why dyspnea occurs so long after beginning to smoke, say 50 to 60 years later. Some patients have even quit smoking several years before symptoms appear. The main reason is that lung function declines slowly with age, even in a nonsmoker.

At approximately age 30, people begin to lose lung function at a rate of 25 to 30 mL/year of forced expiratory volume in 1 second (FEV₁). People who smoke lose lung function at a more rapid rate, approximately 125 mL/year. Because the lungs have a considerable amount of reserve, a large portion must become nonfunctional before symptoms occur. It can take more than 30 years to lose enough lung function to experience symptoms.

When a person quits smoking, the loss in function slows to approximately the rate of a nonsmoker. If smoking has already destroyed a large portion of the lungs, the threshold will be reached, eventually, at a rate of decline of 30 mL/year instead of 125 mL/year. Without quitting, decline would continue at a rate of 125 mL/year.

Quality of life can improve by quitting, even if lung function has already declined. Chronic cough typically begins as a morning cough and slowly progresses to an all-day cough. The cough usually produces small amounts of sputum (less than 60 mL/day) and is clear or whitish but may be discolored. Sputum production decreases when one quits smoking. The progression of the cough's frequency is so slow that a person usually tolerates it for a couple of years before seeing a doctor. Any change in a chronic cough or a new cough in a smoker or patient with COPD should be thoroughly evaluated by a physician. Wheezing is the high-pitched sound of air passing through narrowed airways. A person with COPD may wheeze during an acute exacerbation or chronically. Sometimes the wheezing is heard only at night or with exertion.

Bedoradrine (or MN-221 or KUR-1246) is a pharmaceutical agent that is undergoing human clinical testing, where bedoradrine is administered parenterally. Current indications being investigated include asthma and chronic obstructive pulmonary disease or COPD. Bedoradrine has been described, for example, in U.S. Pat. No. 6,133,266, where it is stated that this 3,4-disubstituted phenylethanolaminotetralincarboxamide derivative can be administered orally or parenterally in the form of appropriate pharmaceutical compositions such as tablets, powders, fine granules, granules, capsules, injections and the like. It is believed, however, that no clinical investigations of bedoradrine involving any other route besides parenteral administration have ever been carried out. Furthermore, prior to the Applicants' investigation, there have been no disclosures, let alone clinical studies, of any possible gender bias in the bioavailability of orally administered bedoradrine or its pharmaceutically acceptable salts.

There also continues to be a need for discovering a treatment that utilizes an oral dosage form and which especially benefits human male patients. Such a treatment would particularly benefit human male patients suffering from COPD because human males make up a great majority of the COPD patient population. Moreover, human male patients have a greater rate of mortality, compared to their human female counterparts, following hospitalization for COPD. In a recent study of gender differences in survival following hospitalization for COPD, it was found that in a study cohort that consisted of 19,260 women and 23,893 men with a mean age of 77 years, 11,245 (58.4%) women and 16,754 (70.1%) men died after cohort entry. Thus, male sex was associated with a significantly increased risk of death (adjusted HR 1.45, 95% CI 1.42 to 1.49) and with a significantly increased risk of re-hospitalization for obstructive airways disease (adjusted HR 1.12, 95% CI 1.09 to 1.15). See, Gonzalez A. V., Suissa S, and Ernst P. Thorax (January 2011) 66(1):38-42, the disclosures of which are incorporated by reference herein in their entirety.

### SUMMARY OF THE INVENTION

The invention arises in part out of the surprising discovery that the bioavailability of bedoradrine or its pharmaceutically acceptable salts, when administered orally or enterally, is higher in human males compared to human females. This discovery is surprising because the bioavailability of bedoradrine or its pharmaceutically acceptable salts, when administered parenterally, is not gender-biased or, if anything, is higher in human females versus human males. This discovery is made even more surprising because orally administered bedoradrine was found to have greater bioavailability in female rats compared to male rats.

Thus, in one aspect, the present invention provides a method of treating a human male patient suffering from a medical condition that is responsive to bedoradrine or a pharmaceutically acceptable salt thereof which comprises enterally, preferably orally, administering an effective amount of bedoradrine or a pharmaceutically acceptable salt thereof to a human male patient in need thereof. In another aspect, the invention provides a method of treating a human male patient suffering from chronic obstructive pulmonary disease (COPD) which comprises enterally, preferably orally, administering an effective amount of bedoradrine or a pharmaceutically acceptable salt thereof to a human male patient suffering from COPD. COPD is a disease that, in part due to its roots to smoking, presents itself more in the human male population. Also provided herein are unit dose pharmaceutically acceptable formulations of bedoradrine or a pharmaceutically acceptable salt thereof, which formulations are suitable for administration in accordance with the present invention.

In a specific embodiment of the invention, a method is provided of decreasing a length of a hospital stay of a human male patient suffering from acute or exacerbated chronic obstructive pulmonary disease (acute COPD or COPD exacerbation) comprising orally administering an effective amount of bedoradrine or a pharmaceutically acceptable salt thereof to a human male patient suffering from an acute COPD or COPD exacerbation who has checked into or been admitted to a hospital.

In another specific embodiment of the invention, a method is provided of improving one or more of FEV₁, FEV₁%, FEV₁ (% predicted), PEFR, or PEFR (% predicted) in a human male patient suffering from an airway constriction or obstructive airways disease comprising orally administering an effective amount of bedoradrine or a pharmaceutically acceptable salt thereof to a human male patient suffering from an airway constriction or obstructive airways disease. In certain embodiment, a patient treated according to the present invention shows a +5%, +10%, +15%, or a +20% improvement in the FEV1, FEV₁% or FEV₁ (% predicted). In certain other embodiment, a patient treated according to the present invention shows a +5%, +10%, +15%, or a +20% improvement in the PEFR or PEFR (% predicted).

The invention also provides a method of reducing the administration of one or more concomitant medications to a human male patient hospitalized for COPD comprising orally administering an effective amount of bedoradrine or a pharmaceutically acceptable salt thereof to a human male patient hospitalized for COPD. In a particular embodiment of the invention, the one or more concomitant medications is selected from the group consisting of 13-agonists other than bedoradrine, antimuscarinics and corticosteroids. More specifically, the one or more concomitant medications is selected from the group consisting of inhaled albuterol, inhaled ipratropium, oral prednisone and intravenous methylprednisolone.

In another embodiment, the medical condition that is responsive to bedoradrine is acute exacerbation of asthma (AEA). In one embodiment, bedoradrine is administered in combination with standard of care treatments for treating AEA. Certain methods of treating AEA and symptoms thereof are described in WO 2011/062984 (incorporated herein in its entirety by reference), which can be adapted by a skilled artisan in view of this disclosure to practice the present invention.

In another preferred embodiment, bedoradrine or a pharmaceutically acceptable salt thereof is administered orally, in a daily amount of 1-50 mg. Within this embodiment, in certain embodiments, bedoradrine or a pharmaceutically acceptable salt thereof is administered orally in a daily amount, more preferably, of 1-30 mg and still more preferably of 5-25 mg. In other preferred embodiments, bedoradrine or a pharmaceutically acceptable salt thereof is administered orally, in a daily amount of 30-50 mg, or more preferably, in a daily amount of 35-45 mg.

Other benefits of the invention include but are not limited to improved respiratory function exhibited by the treated human male patient suffering from the above-described ailments. Such improved respiratory function may be characterized by improvement in one or more of dyspnea, respiratory rate, wheezing, accessory muscle use, supplemental oxygen use, performance on a walking test, or a composite pulmonary function score comprised of two or more of the foregoing measures. Still other benefits are evident from the accompanying disclosure and examples provided herewith.

### BRIEF DESCRIPTION OF THE FIGURES

FIG. 1 graphically illustrates MN-221 plasma concentrations following i.v. (300 μg) or p.o. (600 μg) MN-221 dosing (mean±sd).

FIG. 2 graphically illustrates improved FEV₁ from start of infusion to end of infusion of COPD patients treated with MN-221.

FIG. 3 graphically illustrates improved FEV₁ (% predicted) from start of infusion to end of infusion of COPD patients treated with MN-221.

FIG. 4 graphically illustrates improved PEFR of COPD patients treated with MN-221.

FIG. 5 graphically illustrates improved PEFR (% predicted) of COPD patients treated with MN-221.

FIG. 6 graphically illustrates the efficacy of MN-221 to improve the FEV₁ (% predicted) of AEA patients.

### DETAILED DESCRIPTION OF THE INVENTION

### Definitions

As used herein, and in the appended claims, the singular forms “a,” “an” and “the” include plural references unless the context clearly dictates otherwise.

“Administering” or “Administration of” a drug to a patient (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

“Bedoradrine” or MN-221 refers to bedoradrine, particularly, its hemisulfuric acid salt or bedoradrine sulfate of formula:

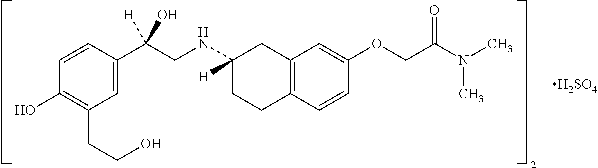

Bedoradrine is synthesized according to methods reported in the literature. See, e.g., the references Yanagi et al. Chem. Pharm. Bull. (Tokyo) (2003) 51(2):221-23 and U.S. Pat. No. 6,133,266 each of which is incorporated herein by reference. Bedoradrine sulfate is a highly selective β-agonist. Other pharmaceutically acceptable salts include, but are not limited to, other mineral acid addition salts, such as hydrochloric acid salts, nitric acid salts, phosphoric acid salts and the like.

“Comprising” shall mean that the compositions and methods include the recited elements, but not exclude others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transitional terms and phrases are within the scope of this invention.

“Effective amount” of bedoradrine or a pharmaceutically acceptable salt thereof is an amount that, when administered to a patient with a medical condition that is responsive to bedoradrine or a pharmaceutically acceptable salt thereof, will have the intended therapeutic effect, e.g., alleviation, amelioration, palliation or elimination of one or more manifestations of the medical condition in the patient. The full therapeutic effect does not necessarily occur by administration of one dose (or dosage), and may occur only after administration of a series of doses. Thus, an effective amount may be administered in one or more administrations.

“FEV₁” refers to the amount of air which can be forcibly exhaled from the lungs in the first second of a forced exhalation. FEV₁% refers to FEV₁ expressed as a percentage of the vital capacity, and is an index for assessing and quantifying airflow limitations. Vital capacity refers to the volume change of the lung between a full inspiration and a maximal expiration. FEV₁ (% predicted) refers to FEV₁ expressed as a percentage of the patient's predicted vital capacity based on the patients age, sex, and body composition. In some embodiments, COPD patients showing FEV₁≧30%<80% are treated according to this invention. In some embodiments, COPD patients showing FEV₁≧30%<70% are treated according to this invention. In some embodiments, COPD patients showing FEV₁%<70% are treated according to this invention.

Peak expiratory flow rate (“PEFR”) (often expressed as L/min) refers to a person's maximum speed of expiration. PEFR measures the airflow through the bronchi and thus the degree of obstruction in the airways. PEFR (% predicted), is the ratio of a patient's PEFR, expressed as percent (%), compared to a calculated normal PEFR for a patient of similar, age, sex, and body composition.

“Pharmaceutically acceptable salt” refers to a salt that is safe for administration to a human to obtain the intended treatment benefit. Pharmaceutically acceptable salts include acid salts such as those derived from inorganic acids, organic acids, and amphoteric compounds. Non limiting examples of acid salts derived from inorganic acids include, chloride, bromide, nitrate, phosphates, diphosphates, sulfate salts, and the like. Non limiting examples of acid salts derived from organic acids include, those derived from acetic acid, methanesulfonic acid, benzenesulfonic acid, p-toluenesulfonic acid, propionic acid, citric acid, succinic acid, tartaric acid, fumaric acid, butyric acid, oxalic acid, malonic acid, maleic acid, lactic acid, malic acid, carbonic acid, glutamic acid, aspartic acid and the like. Non limiting examples of acid salts derived from amphoteric compounds include, those derived from amino acids, preferably from the naturally occurring amino acids well known to the skilled artisan.

“Treating” a medical condition or human male patient refers to taking steps to obtain beneficial or desired results, including clinical results. For purposes of the various aspects and embodiments of the present invention, beneficial or desired clinical results include, but are not limited to, reduction, alleviation, or amelioration of one or more manifestations of or negative effects of medical condition that is responsive to bedoradrine or a pharmaceutically acceptable salt thereof, improvement in one or more clinical outcomes, diminishment of extent of disease, delay or slowing of disease progression, amelioration, palliation, or stabilization of the disease state, and other beneficial results described herein. In one aspect, the invention provides a method of treating a human male patient suffering from a medical condition that is responsive to bedoradrine or a pharmaceutically acceptable salt thereof which comprises enterally, preferably orally, administering an effective amount of bedoradrine or a pharmaceutically acceptable salt thereof to the human male patient who is suffering from a medical condition that is responsive to bedoradrine or a pharmaceutically acceptable salt thereof. In one embodiment, the medical condition that is responsive to bedoradrine or a pharmaceutically acceptable salt thereof is selected from the group consisting of asthma, acute exacerbation of asthma, chronic obstructive pulmonary disease, COPD exacerbations, and other bronchospasms (generally, obstructive airways disease).

### Preferred Embodiments

In one embodiment, the effective amount of bedoradrine or a pharmaceutically acceptable salt thereof administered according to this invention ranges from about 0.1 mg to about 1,000 mg per day. In another embodiment, the effective amount of bedoradrine or a pharmaceutically acceptable salt thereof ranges from about 0.1 mg to about 100 mg per day, about 0.1 mg to about 50 mg per day, or about 1 mg to about 25 mg per day. In yet another embodiment, the effective amount of bedoradrine or a pharmaceutically acceptable salt thereof ranges from about 0.1 mg to about 10 mg per day, preferably, from about 1 mg to about 5 mg per day.

In another embodiment, the effective amount of bedoradrine or a pharmaceutically acceptable salt thereof ranges from about 0.3 mg to about 50 mg per day, about 0.3 mg to about 40 mg per day, about 1.5 mg to about 25 mg per day, about 3 mg to about 15 mg per day, or about 5 mg to about 10 mg per day. In another embodiment, the effective amount of bedoradrine or a pharmaceutically acceptable salt thereof is about 40 mg/day, or ranges from about 30 mg to about 50 mg per day. In another embodiment, the effective amount of bedoradrine or a pharmaceutically acceptable salt thereof ranges from about 0.3 mg to about 15 mg per day. In another embodiment, the effective amount of bedoradrine or a pharmaceutically acceptable salt thereof administered enterally ranges from about 0.5 mg to about 5 mg per day. In another embodiment, about 5 mg to about 25 mg per day, or about 10 mg per day of bedoradrine or a pharmaceutically acceptable salt thereof is administered. In another embodiment, about 1, about 5, about 10, about 15, about 20, about 25, or about 30 mg per day of bedoradrine or a pharmaceutically acceptable salt thereof is administered.

Any means for enteral administration of bedoradrine or a pharmaceutically acceptable salt thereof is suitable with the methods provided herein, including but not limited to orally, by gavage, buccally, or by utilizing an enema or a suppository. In a preferred embodiment, the effective amount of bedoradrine or a pharmaceutically acceptable salt thereof is administered orally. When administered orally, various dosage forms may be appropriate, including but not limited to tablets, powders, fine granules, granules, capsules, or liquids (including syrups, emulsions, suspensions, or solutions)—containing 0.5, 1, 2, 5, 10, 20, or 25 mg of bedoradrine or a pharmaceutically acceptable salt thereof—administered once, twice, thrice, or more often per day. In another embodiment, the effective amount of bedoradrine or a pharmaceutically acceptable salt thereof is administered rectally via a suppository or as an enema. In yet another embodiment, the administration is performed enterally in which about 0.1 mg to less than about 1 mg per day of bedoradrine or a pharmaceutically acceptable salt thereof is administered. The duration of the treatment may vary depending on the nature of the medical condition or the severity of the disease or its symptoms. In COPD, oral bedoradrine treatment from 1 to 14 days is most typical although a chronic (months to years) oral bedoradrine treatment regimen is specifically contemplated in the present invention. In one embodiment, this invention provides chronic oral administration of bedoradrine or a pharmaceutically acceptable salt thereof for treating a male COPD patient. In preferred embodiments, chronic oral administration refers to daily administration from more than 2 weeks to a month or 2 months of treatment. In another embodiment, this invention provides a single daily administration, or 1-3 times daily administration for up to 2 weeks for treating COPD exacerbations or for improving recovery from COPD in an intensive care unit, for a male COPD patient.

Bedoradrine or a pharmaceutically acceptable salt thereof may be formulated in a variety of pharmaceutically acceptable preparations, taking into account, for example, the routes of administration of the compositions. A variety of pharmaceutically acceptable carriers are useful in such formulations, including, without limitation, water, salt solutions, alcohols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, an the likes, as described in further detail below. Freeze dried or lyophilized compositions may also be suitably used. In one embodiment, the pharmaceutically acceptable preparation is a liquid formulation comprising bedoradrine sulfate.

The compounds utilized herein can be formulated in any pharmaceutically acceptable form, including powders, capsules, tablets, suppositories, creams, emulsions, troches, suspensions, liquids, solutions, and the like. Therapeutic compositions containing the compounds utilized herein will ordinarily be formulated with one or more pharmaceutically acceptable ingredients in accordance with known and established practice. In general, tablets are formed utilizing a carrier such as modified starch, alone or in combination with 10% by weight of carboxymethyl cellulose (Avicel). The formulations are compressed at from 1,000 to 3,000 pounds pressure in the tablet forming process. The tablets preferably exhibit an average hardness of about 1.5 to 8.0 kp/cm², preferably 5.0 to 7.5 kp/cm². Disintegration time varies from about 30 seconds to about 15 or 20 minutes.

Formulations for oral use can be provided as hard gelatin capsules wherein the therapeutically active compounds utilized herein are mixed with an inert solid diluent such as calcium carbonate, calcium phosphate or kaolin, or as soft gelatin capsules in which the compounds are mixed with an oleaginous medium, e.g., liquid paraffin or olive oil. Suitable carriers include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like.

Suitable formulations also include sustained release dosage forms, such as those described in U.S. Pat. Nos. 4,788,055; 4,816,264; 4,828,836; 4,834,965; 4,834,985; 4,996,047; 5,071,646; and, 5,133,974, the contents of which are incorporated herein in their entirety by reference.

The compounds utilized herein may be formulated for administration as suppositories. In such a formulation, a low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds utilized herein may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient. A common type of controlled release formulation that may be used for the purposes of the present invention comprises an inert core, such as a sugar sphere, a first layer, coated with an inner drug-containing second layer, and an outer membrane or third layer controlling drug release from the inner layer.

The cores are preferably of a water-soluble or swellable material, and may be any such material that is conventionally used as cores or any other pharmaceutically acceptable water-soluble or water-swellable material made into beads or pellets. The cores may be spheres of materials such as sucrose/starch (Sugar Spheres NF), sucrose crystals, or extruded and dried spheres typically comprised of excipients such as microcrystalline cellulose and lactose.

The substantially water-insoluble material in the first layer is generally a “GI insoluble” or “GI partially insoluble” film forming polymer (dispersed or dissolved in a solvent). As examples may be mentioned ethyl cellulose, cellulose acetate, cellulose acetate butyrate, polymethacrylates such as ethyl acrylate/methyl methacrylate copolymer (Eudragit NE-30-D) and ammonio methacrylate copolymertypes A and B (Eudragit RL30D and RS30D), and silicone elastomers. Usually, a plasticizer is used together with the polymer. Exemplary plasticizers include: dibutylsebacate, propylene glycol, triethylcitrate, tributylcitrate, castor oil, acetylated monoglycerides, acetyl triethylcitrate, acetyl butylcitrate, diethyl phthalate, dibutyl phthalate, triacetin, fractionated coconut oil (medium-chain triglycerides).

The second layer containing the active ingredient may be comprised of the active ingredient (drug) with or without a polymer as a binder. The binder, when used, is usually hydrophilic but may be water-soluble or water-insoluble. Exemplary polymers to be used in the second layer containing the active drug are hydrophilic polymers such as polyvinylpyrrolidone, polyalkylene glycol such as polyethylene glycol, gelatine, polyvinyl alcohol, starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, carboxymethyl hydroxyethyl cellulose, acrylic acid polymers, polymethacrylates, or any other pharmaceutically acceptable polymer. The ratio of drug to hydrophilic polymer in the second layer is usually in the range of from 1:100 to 100:1 (w/w).

Suitable polymers for use in the third layer, or membrane, for controlling the drug release may be selected from water insoluble polymers or polymers with pH-dependent solubility, such as, for example, ethyl cellulose, hydroxypropylmethyl cellulose phthalate, cellulose acetate phthalate, cellulose acetate trimellitate, polymethacrylates, or mixtures thereof, optionally combined with plasticizers, such as those mentioned above.

Optionally, the controlled release layer comprises, in addition to the polymers above, another substance(s) with different solubility characteristics, to adjust the permeability, and thereby the release rate, of the controlled release layer. Exemplary polymers that may be used as a modifier together with, for example, ethyl cellulose include: HPMC, hydroxyethyl cellulose, hydroxypropyl cellulose, methylcellulose, carboxymethylcellulose, polyethylene glycol, polyvinylpyrrolidone (PVP), polyvinyl alcohol, polymers with pH-dependent solubility, such as cellulose acetate phthalate or ammonio methacrylate copolymer and methacrylic acid copolymer, or mixtures thereof. Additives such as sucrose, lactose and pharmaceutical grade surfactants may also be included in the controlled release layer, if desired.

The compounds utilized herein can be formulated as aqueous suspensions in admixture with pharmaceutically acceptable excipients such as suspending agents, e.g., sodium carboxymethyl cellulose, methylcellulose, hydroxypropylmethyl cellulose, sodium alginate, polyvinylpyrrolidone, gum tragacanth and gum acacia; dispersing or wetting agents such as naturally occurring phosphatide, e.g., lecithin, or condensation products of an alkaline oxide with fatty acids, e.g., polyoxyethylene stearate, or condensation products of ethylene oxide with long chain aliphatic alcohols, e.g, heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. Such aqueous suspensions can also contain one or more preservatives, e.g., ethyl- or -n-propyl-p-hydroxy benzoate, one or more coloring agents, one or more flavoring agents and one or more sweetening agents, such as glycerol, sorbitol, sucrose, saccharin or sodium or calcium cyclamate.

Other forms suitable for oral administration include liquid form preparations including emulsions, syrups, elixirs, aqueous solutions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizing, and thickening agents. Solid form preparations may contain, in addition to the active component, colorants, flavors, stabilizers, buffers, artificial and natural sweeteners, dispersants, thickeners, solubilizing agents, and the like.

Also provided herein are unit dosage forms of the formulations. In such forms, the formulation is subdivided into unit dosages containing appropriate quantities of the active component (i.e. bedoradine or a pharmaceutically acceptable salt of thereof). The unit dosage form can be a packaged preparation, the package containing discrete quantities of preparation, such as, preferably, packeted tablets and capsules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

The present invention having been described in summary and in detail, is illustrated and not limited by the following examples.

### EXAMPLES

### Example 1

### A Phase I, Open-label, Single-Dose Crossover Test to Evaluate the Oral Bioavailability, Safety, and Tolerability of Bedoradrine Sulfate in Healthy Volunteers

This example demonstrates the enhanced oral bioavailability of bedoradrine sulfate based on pharmacokinetic analysis of bedoradrine sulfate administration in cross-over single oral and i.v. administration of bedoradrine sulfate to male and female healthy subjects under fasted conditions.

### Methodology

a. Test Design

The test was an open-label, randomized, crossover, single dose test of 600 μg (0.6 mg) bedoradrine sulfate in liquid form administered orally and of 300 μg (0.3 mg) bedoradrine sulfate administered by i.v. infusion. All subjects were in an overnight fasting state. Four male and four female subjects were enrolled in the test and received two administration sequences:

Day 1: n=4 received 15-minute intravenous infusion of bedoradrine sulfate (300 μg), the other 4 received an oral administration of bedoradrine sulfate (600 μg);
Day 8: the 4 subjects administered i.v. MN-221 on Day 1, received oral bedoradrine sulfate and the 4 subjects administered orally with MN-221 on Day 1, receive i.v. bedoradrine sulfate.

Subjects were domiciled in the clinical research unit (CRU) for two nights, beginning on Day minus one (−1), one day prior to dosing. First dosing occurred in the morning of Day 1. Subjects were discharged from the CRU approximately 24 hours after dosing (Day 2). There was a one-week washout between doses and subjects returned to the CRU (Day 7) and were domiciled for two days. On the morning of Day 8, subjects received the opposite dosing state (from Day 1) and subjects were then discharged from the CRU approximately 24 hours after dosing (Day 9).

TABLE 1
Subject Dosing Configuration

| Subject # | Gender | Day 1 | Day 8 |
|---|---|---|---|
| 001 | F | i.v. | p.o. |
| 002 | F | i.v. | p.o. |
| 003 | M | p.o. | i.v. |
| 004 | M | i.v. | p.o. |
| 005 | M | i.v. | p.o. |
| 006 | F | p.o. | i.v. |
| 007 | M | p.o. | i.v. |
| 008 | F | p.o. | i.v. |

Blood samples for plasma selection and bioanalysis were collected on the dosing days before dosing (baseline) and at 0.25, 1, 1.5, 2, 3, 4, 6, 9, 12, and 24 hr after dosing.

b. Bioanalytical Methodology

The bioanalytical results were obtained following HPLC/MS/MS procedures. The limit of quantitation (LLOQ) for these methods was 30.3 picogram (pg)/mL.

c. Pharmacokinetic and Statistical Analysis

Plasma MN-221 concentrations were tabulated separately by subject and sampling time and summarized by descriptive statistics. Descriptive statistics included the sample size, mean, standard deviation, median, range, and the coefficient of variation (CV %). Mean plasma concentration-time profiles were illustrated graphically. All plasma concentrations below the limit of quantitation (BLQ) values occurring at predose and up to the first measurable concentration may otherwise be imputed as 1 pg/mL in some analyses or LLOQ. Pharmacokinetic parameters were calculated from individual plasma concentrations-time data by model-independent analysis methods using WinNonlin™. The following parameters were calculated: Cₘₐₓ (observed maximum plasma concentration), tₘₐₓ (observed time of maximum plasma concentration), t_{1/2} (terminal half-life calculated as ln(2)/λ_{z}), AUC₀₋₂₄ (area under the plasma concentration-time curve calculated using the linear trapezoidal rule from time zero to time 24 hr), vₛₛ (volume of distribution at steady state), Cl (clearance), and Oral F % (% oral bioavailability).

All pharmacokinetic parameters were summarized using descriptive statistics that included the sample size, mean, standard deviation, median, range, and coefficient of variation. Select t-test or non-parametric statistical analysis was optional.

### Results

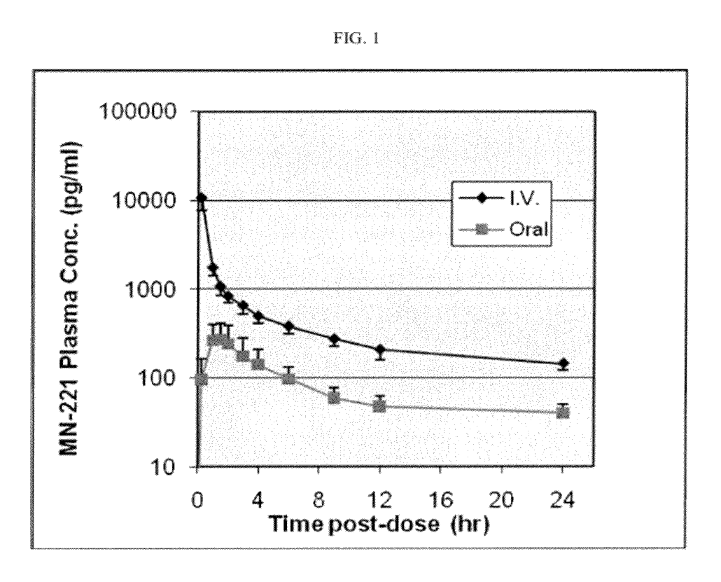

The individual subject plasma concentrations of bedoradrine sulfate are listed in Table 2.1. Table 2.2 and FIG. 1 provide the time course of the average and standard deviation plasma concentrations.

TABLE 2.1
Individual Subject MN-221 Plasma Concentrations

| | Subject | Time Point (hr) | | | | | | | | | | |
|---|---|---|---|---|---|---|---|---|---|---|---|---|
| | # | 0 (BL) | 0.25 | 1 | 1.5 | 2 | 3 | 4 | 6 | 9 | 12 | 24 |
| Day 1 | 001 | <30.3 | 12142.1 | 2210.5 | 1413.9 | 1031.9 | 746.7 | 525.2 | 421.6 | 274.5 | 218.0 | 119.0 |
| | 002 | <30.3 | 12474.8 | 1648.3 | 1143.9 | 793.1 | 627.9 | 486.5 | 422.4 | 269.6 | 175.1 | 150.6 |
| | 003 | <30.3 | 31.3 | 500.0 | 449.7 | 494.0 | 383.7 | 244.3 | 143.5 | 73.0 | 56.2 | 58.0 |
| | 004 | <30.3 | 6759.9 | 2160.0 | 1295.7 | 755.3 | 762.4 | 516.2 | 329.0 | 249.1 | 131.4 | 105.6 |
| | 005 | <30.3 | 7750.6 | 1647.3 | 1017.2 | 779.0 | 591.2 | 477.3 | 375.7 | 267.8 | 218.3 | 147.6 |
| | 006 | <30.3 | 162.9 | 94.7 | 69.9 | 93.4 | 87.8 | 74.6 | 41.3 | 31.3 | <30.3 | <30.3 |
| | 007 | <30.3 | <30.3 | 210.2 | 284.9 | 302.8 | 234.9 | 219.4 | 86.9 | 41.5 | 34.5 | 37.9 |
| | 008 | <30.3 | 212.3 | 170.8 | 113.3 | 93.1 | 91.8 | 124.7 | 109.8 | 66.7 | 40.8 | <30.3 |
| Day 8 | 001 | <30.3 | 81.4 | 270.7 | 299.5 | 188.5 | 143.9 | 104.2 | 76.3 | 49.2 | 51.8 | 43.7 |
| | 002 | <30.3 | 55.7 | 153.2 | 209.1 | 125.3 | 67.6 | 55.4 | 81.3 | 55.6 | 44.1 | 33.9 |
| | 003 | <30.3 | 6871.2 | 1162.0 | 728.9 | 629.6 | 410.8 | 350.1 | 242.7 | 203.3 | 162.0 | 157.0 |
| | 004 | <30.3 | 55.3 | 280.1 | 306.8 | 289.4 | 207.8 | 161.8 | 123.7 | 62.5 | 46.4 | 44.5 |
| | 005 | <30.3 | 144.2 | 422.9 | 443.9 | 364.1 | 184.0 | 143.7 | 122.3 | 92.9 | 78.9 | 45.4 |
| | 006 | <30.3 | 14823.1 | 1626.9 | 972.0 | 881.7 | 627.7 | 508.5 | 408.1 | 259.4 | 239.7 | 154.3 |
| | 007 | <30.3 | 12300.5 | 1716.6 | 960.4 | 862.6 | 633.5 | 457.8 | 389.8 | 295.5 | 241.3 | 166.9 |
| | 008 | <30.3 | 12813.5 | 1774.0 | 1032.1 | 907.1 | 801.1 | 633.3 | 452.0 | 364.7 | 278.7 | 158.2 |

TABLE 2.2
Average and Standard Deviation Plasma Concentrations vs Time

| | |
|---|---|
| i.v. | oral |

| Time (hr) | mean | Sd | mean | sd |
|---|---|---|---|---|
| 0.0 | BLQ | | BLQ | |
| 0.3 | 10742.0 | 3119.7 | 96.6 | 68.0 |
| 1.0 | 1743.2 | 330.2 | 262.8 | 138.2 |
| 1.5 | 1070.5 | 212.8 | 272.1 | 138.0 |
| 2.0 | 830.0 | 119.6 | 243.8 | 144.0 |
| 3.0 | 650.2 | 123.5 | 175.2 | 103.7 |
| 4.0 | 494.3 | 78.8 | 141.0 | 66.1 |
| 6.0 | 380.1 | 66.6 | 98.1 | 32.8 |
| 9.0 | 273.0 | 45.6 | 59.1 | 19.3 |
| 12.0 | 208.1 | 48.4 | 47.8 | 15.2 |
| 24.0 | 144.9 | 21.2 | 40.4 | 9.5 |

After i.v. administration, the bedoradrine sulfate plasma concentrations peaked at the first sampling time point (end-of-infusion, 0.25 hr) and exhibited curvelinear decay with time. Standard deviation did not exceed 30% of the mean value at any time point for i.v. dosing. After oral administration, the bedoradrine sulfate plasma concentrations increased and reached maximal concentrations within 2 hr (median 1.5 hr) after dosing. The plasma concentrations declined thereafter. Whole group standard deviation ranged 23-70% of the mean across the sampling time points. Pharmacokinetic analysis results are summarized collectively in Table 2.3 and for individual patients (or i.v and oral dosing) in Tables 2.4 and 2.5.

TABLE 2.3
Pharmacokinetic Parameters of Bedoradrine Sulfate Obtained After Single Intravenous (I.V.) or
Oral (P.O.) Dose Administration of Bedoradrine Sulfate to Male and Female Healthy Subjects

| | | | | | AUC | | | | |
|---|---|---|---|---|---|---|---|---|---|
| | Dose | | Cmax | Tmax | 0-24 h | CL | Vss | t½ | Oral |
| Route | (μg) | Function | (pg/mL) | (hr) | (pg*hr/mL) | (L/hr) | (L) | (hr) | F % |
| I.V. | 300 | mean | 10741.9 | 0.25 | 13209.5 | 19.3 | 221.6 | 12.2 | NA |
| | | median | 12221.0 | 0.25 | 14108.5 | 18.4 | 206.5 | 12.5 | NA |
| | | sd | 3119.8 | 0.0 | 2385.0 | 4.1 | 66.3 | 2.9 | NA |
| | | CV % | 29.0 | 0.0 | 18.1 | 21.3 | 29.9 | 23.6 | NA |
| | | range | 6760-14823 | NA | 9308-15488 | 15.1-25.5 | 149-325 | 7.4-14.6 | NA |
| P.O. | 600 | mean | 304.6 | 1.2 | 1942.1 | NA | NA | 16.1 | 8.0 |
| | | median | 301.0 | 1.5 | 1848.5 | NA | NA | 15.2 | 6.4 |
| | | sd | 116.8 | 0.6 | 656.9 | NA | NA | 4.7 | 4.3 |
| | | CV % | 38.3 | 53.7 | 33.8 | NA | NA | 29.3 | 53.8 |
| | | Range | 163-500 | 0.25-2 | 1048-3021 | NA | NA | 11-23 | 3.4-16.2 |

NA = Not Applicable

TABLE 2.4
Individual Subject PK Parameters (I.V. Dosing)

| | | | | | AUC | | | |
|---|---|---|---|---|---|---|---|---|
| Subject | | Dose | Cmax | Tmax | 0-24 h | CL | Vss | t½ |
| # | Gender | (μg) | (pg/mL) | (hr) | (pg*hr/mL) | (L/hr) | (L) | (hr) |
| 1 | F | 300 | 12142 | 0.25 | 14695 | 17.8 | 163 | 12.7 |
| 2 | F | 300 | 12475 | 0.25 | 13874 | 18.9 | 149 | 9.3 |
| 3 | M | 300 | 6871 | 0.25 | 9308 | 25.5 | 299 | 10.9 |
| 4 | M | 300 | 6760 | 0.25 | 10670 | 25.4 | 188 | 7.4 |
| 5 | M | 300 | 7750 | 0.25 | 11571 | 19.4 | 325 | 14.6 |
| 6 | F | 300 | 14823 | 0.25 | 15488 | 15.1 | 225 | 14.6 |
| 7 | M | 300 | 12300 | 0.25 | 14343 | 15.8 | 257 | 15.8 |
| 8 | F | 300 | 12814 | 0.25 | 15727 | 16.2 | 167 | 12.2 |

TABLE 2.5
Individual Subject PK Parameters (Oral Dosing)

| | | | | | AUC | | |
|---|---|---|---|---|---|---|---|
| Subject | | Dose | Cmax | Tmax | 0-24 h | t½ | Oral |
| # | Gender | (μg) | (pg/mL) | (hr) | (pg*hr/mL) | (hr) | F % |
| 1 | F | 600 | 299 | 1.5 | 1790 | 11.6 | 6.1 |
| 2 | F | 600 | 209 | 1.5 | 1377 | 23.3 | 5.0 |
| 3 | M | 600 | 500 | 1 | 3021 | 18.6 | 16.2 |
| 4 | M | 600 | 307 | 1.5 | 2135 | 15.7 | 10.0 |
| 5 | M | 600 | 444 | 1.5 | 2680 | 14.6 | 11.6 |
| 6 | F | 600 | 163 | 0.25 | 1048 | 12.5 | 3.4 |
| 7 | M | 600 | 303 | 2 | 1907 | 22 | 6.6 |
| 8 | F | 600 | 212 | 0.25 | 1579 | 10.8 | 5.0 |

Following 0.3 mg bedoradrine sulfate intravenous dosing, plasma bedoradrine sulfate levels achieved a maximal concentration of nearly 11 ng/mL which was observed at the end-of-infusion for all subjects. Plasma AUC over the test period (0-24 hr) was approximately 13 ng*hr/mL. These parameters as well as clearance, volume of distribution, and elimination half-life was similar to, or predictable from, prior bedoradrine sulfate clinical trial PK results.

Following 0.6 mg MN-221 oral dosing, average peak plasma MN-221 levels were about 0.3 ng/mL or >30-fold lower than the Cmax observed after 0.3 mg i.v. dosing. The AUC following oral dosing was about 7-fold lower than that observed at the i.v. dose. Accordingly, the average oral bioavailability was about 8%. Peak plasma levels upon oral dosing was observed at approximately 1.5 hr post-dose. The elimination half-life ranged 11-23 hr with a mean of 16 hr which was slightly longer than that observed following i.v. dosing.

Interestingly, upon analysis of individual subject plasma PK profiles, there appeared to be a gender effect for bedoradrine sulfate, particularly following oral dosing. Accordingly, the PK parameters for i.v. and oral dosing were re-grouped by gender and are summarized below.

TABLE 2.6
Gender Relationship for PK Parameters Following Oral Bedoradrine Sulfate Dosing

| | | Cmax | | | AUC0-24 h | | | oral F % | | |
|---|---|---|---|---|---|---|---|---|---|---|
| Gender | Route | mean | sd | range | mean | sd | range | mean | sd | range |
| M | i.v. | 8420 | 2624 | 6760-12300 | 11473 | 2127 | 9308-14343 | — | — | — |
| F | i.v. | 13064 | 1205 | 12142-14823 | 14946 | 840 | 13874-15488 | — | — | — |
| M | p.o. | 389 | 99 | 303-500 | 2436 | 507 | 1907-3021 | 11.1 | 4.0 | 6.6-16.2 |
| F | p.o. | 221 | 57 | 163-299 | 1449 | 316 | 1048-1790 | 4.9 | 1.1 | 3.4-6.1 |

Notably, the ranges for Cmaxand AUC in the four males vs four females following oral dosing did not overlap, with both parameters higher for M vs F. Indeed, the derived oral bioavailability values likewise did not overlap between genders with the oral F % in men being two-fold greater than in women. Statistical analysis by t-test confirmed the difference with p<0.05. Such an apparent gender effect was not observed upon i.v. dosing. Actually, a trend was evident wherein females showed higher average Cₘₐₓ and AUC values as compared to males.

### Conclusions

This test demonstrates an oral administration and PK determination of bedoradrine sulfate in humans. Human males and females were administered bedoradrine sulfate solutions at 0.3 mg i.v. and 0.6 mg p.o. PK analysis of plasma concentration vs time data revealed i.v. PK profiles consistent with, or expected from, prior bedoradrine sulfate trials. Interestingly, oral PK analyses revealed low oral bioavailability (group mean=8%), albeit good half-life (group average=16 hr). However, in this test, in these healthy subjects, and at this oral dose level, there appeared to be a gender effect for plasma levels following oral dosing: Males exhibited higher Cmaxand AUC levels—and accordingly, greater oral bioavailability—than females. Given that a trend in the reverse manner was evident with i.v. bedoradrine sulfate dosing, without being bound by mechanism or theory, altered gastric absorption and/or GI metabolism in M vs F may be responsible the surprising gender bias of bioavailability observed. Oral F % determined in mammals varied widely and unpredictably and was ≦3% in rats and rabbits and 39% in dogs. Oral F % in human males appear to fall in-between these numbers.

### Example 2

### A Clinical Test to Evaluate the Oral Bioavailability, Safety, and Tolerability of Bedoradrine Sulfate in Healthy Volunteers Administered a Solid Formulation of Bedoradrine

This example tests the enhanced oral bioavailability of bedoradrine sulfate based on pharmacokinetic analysis of bedoradrine sulfate administration in cross-over single oral (as a capsule or a tablet) and i.v. administration of bedoradrine sulfate to male and female healthy subjects under fasted conditions.

### Methodology

a. Test Design

The test is an open-label, randomized, crossover, single dose test of 600 μg (0.6 mg) bedoradrine sulfate in solid form (a tablet or a capsule) administered orally and of a sterile solution of 300 μg (0.3 mg) bedoradrine sulfate administered by i.v. infusion. All subjects are in an overnight fasting state. Four male and four female subjects are enrolled in the test and receive two administration sequences:

Day 1: n=4 receives a 15-minute intravenous infusion of bedoradrine sulfate (300 μg), the other 4 receive an oral administration of bedoradrine sulfate (600 μg);
Day 8: the 4 subjects administered i.v. MN-221 on Day 1, receive oral bedoradrine sulfate and the 4 subjects administered orally with MN-221 on Day 1, receive i.v. bedoradrine sulfate.

Subjects are domiciled in the clinical research unit (CRU) for two nights, beginning on Day minus one (−1), one day prior to dosing. First dosing occurs in the morning of Day 1. Subjects are discharged from the CRU approximately 24 hours after dosing (Day 2). There is a one-week washout between doses and subjects returns to the CRU (Day 7) and are domiciled for two days. On the morning of Day 8, subjects receive the opposite dosing state (from Day 1) and subjects are then discharged from the CRU approximately 24 hours after dosing (Day 9).

TABLE 1
Subject Dosing Configuration

| Subject # | Gender | Day 1 | Day 8 |
|---|---|---|---|
| 001 | F | i.v. | p.o. |
| 002 | F | i.v. | p.o. |
| 003 | M | p.o. | i.v. |
| 004 | M | i.v. | p.o. |
| 005 | M | i.v. | p.o. |
| 006 | F | p.o. | i.v. |
| 007 | M | p.o. | i.v. |
| 008 | F | p.o. | i.v. |

Blood samples for plasma selection and bioanalysis are collected on the dosing days before dosing (baseline) and at 0.25, 1, 1.5, 2, 3, 4, 6, 9, 12, and 24 hr after dosing.

b. Bioanalytical Methodology

The bioanalytical results are obtained following HPLC/MS/MS procedures. The limit of quantitation (LLOQ) for these method, for example, may be 30.3 picogram (pg)/mL.

c. Pharmacokinetic and Statistical Analysis

Plasma MN-221 concentrations are tabulated separately by subject and sampling time and summarized by descriptive statistics. Descriptive statistics include the sample size, mean, standard deviation, median, range, and the coefficient of variation (CV %). Mean plasma concentration-time profiles are illustrated graphically. All plasma concentrations below the limit of quantitation (BLQ) values occurring at predose and up to the first measurable concentration may otherwise be imputed as 1 pg/mL in some analyses or LLOQ. Pharmacokinetic parameters are calculated from individual plasma concentrations-time data by model-independent analysis methods using WinNonlin™. The following parameters are calculated: Cₘₐₓ (observed maximum plasma concentration), tₘₐₓ (observed time of maximum plasma concentration), t_{1/2} (terminal half-life calculated as ln(2)/λ_{z}), AUC₀₋₂₄ (area under the plasma concentration-time curve calculated using the linear trapezoidal rule from time zero to time 24 hr), vₛₛ (volume of distribution at steady state), Cl (clearance), and Oral F % (% oral bioavailability).

All pharmacokinetic parameters are summarized using descriptive statistics that includes the sample size, mean, standard deviation, median, range, and coefficient of variation. Select t-test or non-parametric statistical analysis is optionally performed. The oral bioavailability is determined as described in Example 1 above.

### Example 3

### Treatment of COPD Using Oral Bedoradrine Sulfate

Human male subjects presenting themselves in an emergency room setting with COPD exacerbations or are already admitted into the ICU with COPD-related bronchioconstriction are screened to determine if they satisfy certain COPD exacerbations inclusion criteria. Patients that satisfy the inclusion criteria (male 40-70 years of age, inclusive; history of physician-diagnosed (e.g., by clinical history, >10 pack year history of smoking, physical examination, and spirometry) COPD treated for ≧3 months prior to Screen Visit 1; FEV₁≧30%<80%) are treated with oral bedoradrine sulfate at doses ranging 0.1 to 20 mg once or twice, each ˜12 hrs for one day (e.g., COPD exacerbations) or for 2-14 days (e.g., ICU or other-ward hospitalized patients). The inclusion criteria are:

COPD and one of the following:
1. History of hospitalization for COPD exacerbation, and/or
2. Currently on supplemental oxygen, and/or
3. History of evaluation for lung transplant or LVRS, and/or
4. >3 months post-LVRS and/or >3 mos diagnosis of COPD
5. Current or former smoker, >10 pack-yr. smoking history
6. FEV₁</=70%; FEV₁/FVC <80%
7. Life expectancy of >6 months
   The exclusion criteria are:
1. <10 pack-yr. smoking history, OR
2. Diagnosis of pulmonary fibrosis, bronchiectasis, mediastinal mass, or presence of a pulmonary mass, OR
3. Asthma (but only because of the study objectives to illustrate utility in human male COPD patients; the invention is otherwise also useful in the treatment of human male patients suffering from asthma or acute exacerbations of asthma), OR
4. Female gender

Primary outcome includes an improvement in FEV₁. Secondary outcomes of the treatment include a reduction in length of hospitalization, respiratory rate, accessory muscle use, supplemental oxygen use, an improvement in Composite Pulmonary Function Score (CPFS), including, changes in dyspnea (e.g. Borg CR10 scale) and six-minute walk test; an improvement in BODE Index, including need for intubation or mechanical ventilation and amount of rescue β-agonist use; and oxygen requirements. Additional exclusion criteria may include human male patients recently hospitalized and having received antibiotics for more than 24 h, or patients on long-term steroids

### Example 4

### Additional Clinical Studies Involving Human Male COPD Patients Overall Study Design and Plan

This is a randomized, double-blind, placebo-controlled, dose escalation study in human male subjects diagnosed with stable moderate to severe COPD, which will be conducted at Clinical Research Units (CRUs) in the U.S. Subjects may be screened to demonstrate an improvement in FEV₁% predicted after bronchodilator treatment of at least 10% at Screen Visit 1. Alternatively, moderate to severe COPD patients may be enrolled without albuterol response profiling. The subject's degree of dyspnea will be captured on the British Medical Research Council (MRC) questionnaire, and severity will be determined by the Global Initiative for Chronic Obstructive Lung Disease (GOLD) spirometric criteria (Appendix 2). Subjects meeting entry criteria at Screen Visit 1 will be asked to return to the CRU for Screen Visit 2 within 14 days of Visit 1. Subjects confirming entry criteria including degree of COPD severity by spirometry at Screen Visit 2 will be randomized to receive either bedoradrine sulfate or placebo. Serial spirometry will be performed over the 8 hour treatment period after initiation of study drug administration. Subjects will be discharged from the CRU after completing the Hour 8 study procedures and asked to return approximately 24 hours after initiation of study drug for follow up safety assessments including spirometry. A study diary will be provided to each subject upon discharge from the CRU to complete as instructed and return it to the site at the 24 hour Follow-up Visit. There will be three dose levels and each will include approximately 16 subjects randomized to receive either bedoradrine sulfate or placebo in 3:1 ratio (12 subjects receive bedoradrine sulfate:4 subjects receive placebo). The initial dose group will be randomized to receive total dose of 600 μg bedoradrine sulfate or placebo. Two subsequent dose levels planned are 1200 μg and 2000 μg and 5000 ug of bedoradrine sulfate or placebo. Upon approximately 16 subjects completing each dose group a risk/benefit evaluation will be performed by the study's Safety Review Committee prior to escalating to the next dose level.

Safety and efficacy will be monitored throughout the treatment period. Blood samples for PK parameters and metabolite identification will be obtained. Inclusion and exclusion criteria are as described above for Example 2.

After oral drug administration, measures of bronchodilation and clinical improvement may be measured at various intervals for several hours. If significant improvement in FEV1, PEFR, RR, accessory muscle use, ease of walking, etc. are observed in the human male COPD patients, then it is concluded that they benefitted from oral bedoradrine treatment.

### Example 5

### Treating COPD by Administering MN-221

This example provides certain preferred oral dosages for treating male COPD patients according to this invention. A randomized, double-blind, placebo-controlled phase Ib clinical test evaluated MN-221 in 48 moderate-to-severe COPD patients who received a 1 hour intravenous (i.v.) infusion of MN-221 at three different escalating dose levels (300 micrograms, 600 micrograms, or 1200 micrograms) or placebo.

Subjects included in this test showed the following history an/or COPD symptoms:

(a) diagnosed (e.g., by clinical history, 20 pack/year smoking history, physical examination [PE], and spirometry) COPD treated for ≧3 months prior to Screen Visit 1;
(b) ≧30% FEV₁<80% and FEV₁/FVC (forced vital capacity) ratio<0.7 at Screen Visits 1 and 2; and
(c) increase in FEV₁ of 12%, over the pre-albuterol FEV₁ within 30 min after inhalation of up to 4 puffs of albuterol delivered by Metered Dose Inhaler (MDI) with spacer at the Screen Visit 1.

48 subjects with stable, moderate-to-severe chronic obstructive pulmonary disease were administered MN-221. The following does were administered. 10 μg/min for 15 min+3.3 μg/min for 45 min (1-hr infusion total dose 300 μg) or placebo; 20 μg/min for 15 min+6.67 μg/min for 45 min (1-hr infusion total dose 600 μg) or placebo; and 40 μg/min for 15 min+13.3 μg/min for 45 min (1-hr infusion total dose 1,200 μg) or placebo.

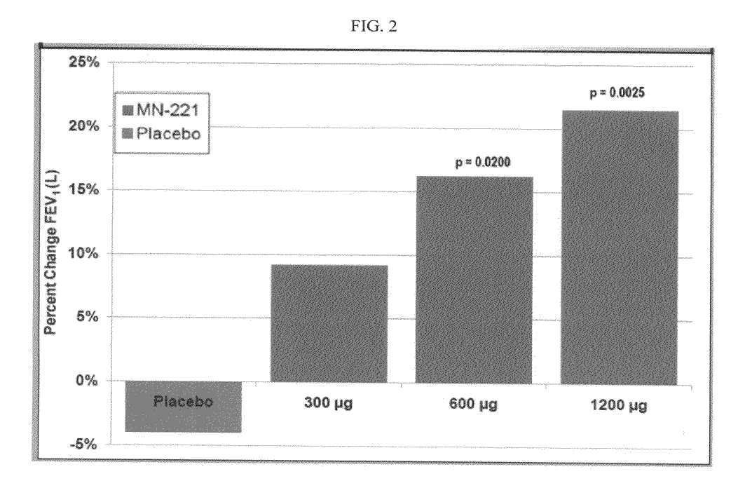

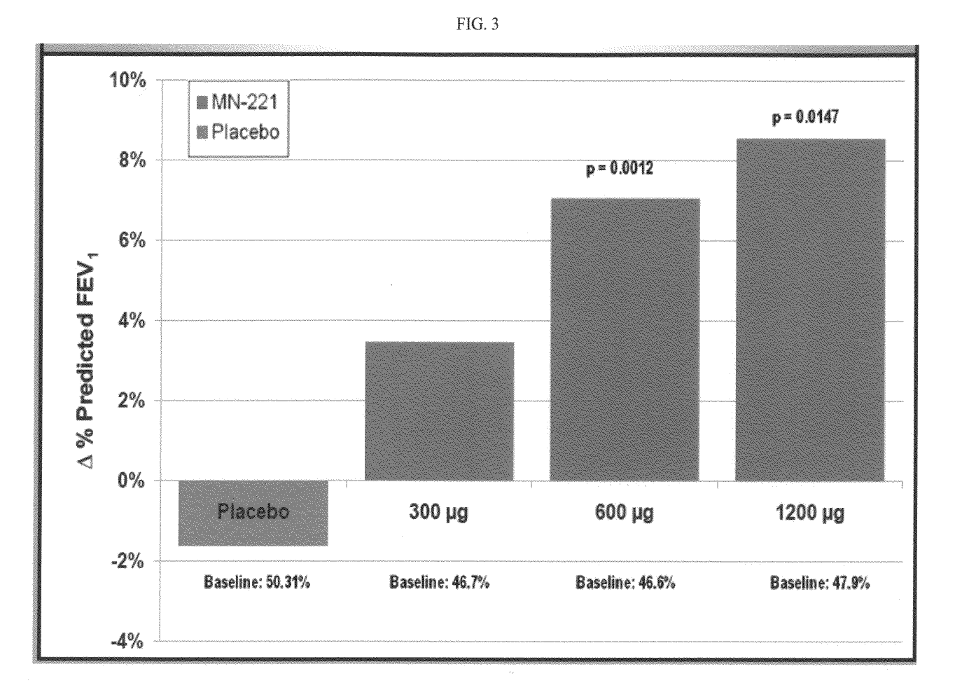

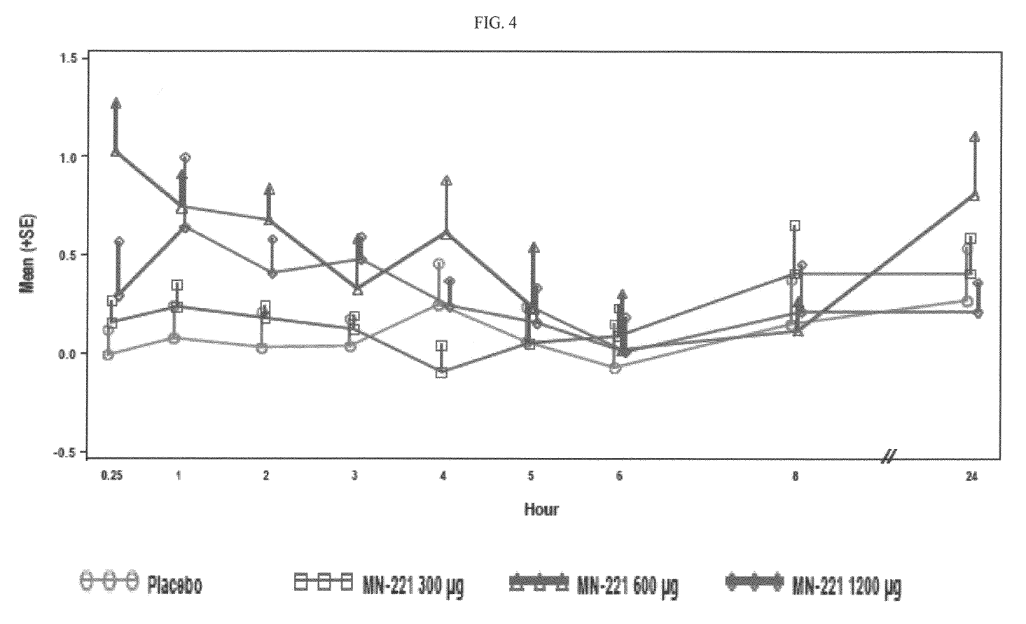

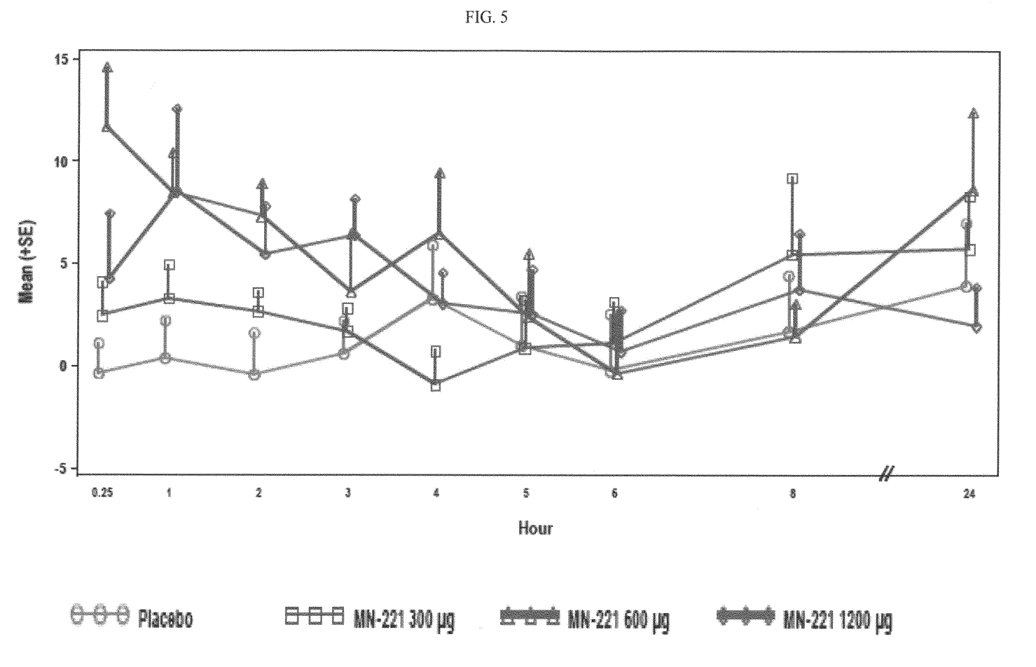

All doses of MN-221 produced substantial improvement in FEV₁ as compared to the baseline and placebo. At the end of the one hour infusion, FEV₁ increased as compared to baseline by an average of 21.5% (p=0.0025) for the 1200 microgram dose, 16.2% (p=0.020) for the 600 microgram dose, and 9.2% (p=NS) for the 300 microgram dose compared to a decrease of 4.0% for the placebo. See also, FIG. 2. The FEV₁ (%, predicted) also improved in the MN-221 treated subjects (FIG. 3). Improved PEFR (Liter(L)/sec) or PEFR (% predicted), also demonstrated the efficacy of MN-221 to improve negative effects of COPD. See, FIGS. 4 and 5. Mean changes in PEFR (L/sec) for the MN-221 (600 and 1200 μg) groups were greatest during 1-3 hr post infusion and were higher than the all placebo group. Mean changes in PEFR (% predicted) for MN-221 (600 and 1200 μg) groups were greatest in first 3-4 hr post-infusion and were higher than the all placebo group. MN-221 was well tolerated by all patients who received infusions of MN-221.

Based on these results, i.v. administration of about 0.3 mg-about 1.2 mg of MN-221, particularly, about 0.6 mg-about 1.2 mg of MN-221 effectively treated COPD. For all practical purposes, MN-221 administered i.v. is considered 100% bioavailable. Based on Example 1, Table 2.6, MN-221's oral bioavailability in male humans is about 7%-16%. Therefore, it is contemplated that in a preferred embodiment, about 1.5 mg to about 25 mg MN-221 administered orally is useful for treating stable COPD or COPD exacerbations and negative effects thereof, in male COPD patients.

### Example 6

### Determining Therapeutically Effective Dosage of Orally Administered MN-221 for Treating COPD

This example provides certain preferred oral dosages for treating male COPD patients according to this invention. Initially, the efficacy and safety of administering intravenous MN-221 in combination with standard of care (SOC) for treating acute exacerbations of asthma (AEA) in an emergency department (ED) setting is determined. The testing was performed in a randomized, placebo-controlled, dose-escalating, setting. Twenty nine patients received SOC (nebulized albuterol and ipratropium plus oral corticosteroid). Patients with FEV₁≦55% of predicted were randomized to MN-221 (240 or 450 μg infused over 15 min or 1080 μg over 2 hr) or Placebo. MN-221 was administered as follows: 16 μg/min for 15 min (total dose of 240 μg); 30 μg/min for 15 min (total dose of 450 μg); 16 μg/min for 15 min+8 μg/min for 105 min (total dose of 1,080 μg); and 2 patients received total dose of 1,995 μg. Safety, efficacy and PK parameters were monitored hourly throughout a 5 hr treatment period and a 24 hr follow-up visit. Nebulized albuterol (2.5 mg) and/or ipratropium (0.5 mg) was available hourly during screening and treatment phases. Efficacy endpoints included spirometry, dyspnea indexing, albuterol use, and hospitalization rates. The testing was completed with 13 patients (n=13) in SOC+Placebo and 16 patients (n=16) receiving SOC+MN-221; 5 at 240 μg, 6 at 450 μg, and 5 intended for a 1080 μg dose. Patients received Standard of Care (SOC) treatment in addition to adjunctive treatment with MN-221 or placebo. MN-221 was well-tolerated.

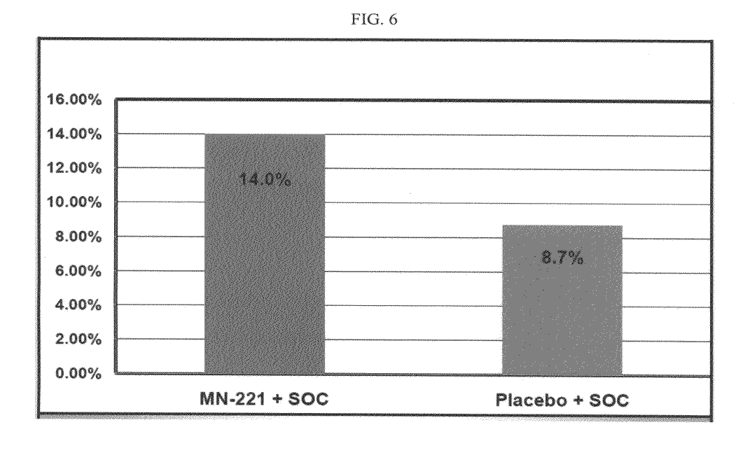

MN-221 treated patients demonstrated reduced hospitalization rate: 4/16 (25%) in the all MN-221 group compared to 17/13 (54%) in Placebo arm were hospitalized. The MN-221 treated patients also demonstrated improved FEV₁ (% predicted), which was elevated in all the MN-221 groups (change from baseline in AUC1-5 hr was 43% higher in the all MN-221 group vs. Placebo). An improved FEV₁ (% predicted) was also observed in patients administered MN-221. See, FIG. 6. The majority of adverse events were mild to moderate in intensity and side effects were lower in the all MN-221 group vs. Placebo and were asthma related side effects. No abnormal electrocardiograph (ECG) findings were observed and heart rate was minimally elevated in the all MN-221 group relative to Placebo. MN-221 adjunctive to standard therapy for severe acute asthma exacerbations was safe and appeared to provide additional clinical benefit.

Based on these results, i.v. infusion of about 0.24 mg-about 1 mg of MN-221 effectively treated AEA. As discussed above, for all practical purposes, MN-221 infused intravenously is 100% bioavailable. Therefore, it is contemplated that in a preferred embodiment, about 1.5 mg-about 20 mg MN-221 administered orally is useful for treating COPD, in male COPD patients.

### Example 7

### Treatment of COPD in Male Patients by Orally Administering a Solid Dosage Form of Bedoradrine

Males patients included in this test show the following history an/or COPD symptoms:

(a) diagnosed (e.g., by clinical history, 20 pack/year smoking history, physical examination [PE], and spirometry) COPD treated for ≧3 months prior to Screen Visit 1;
(b) ≧30% FEV₁<80% and FEV₁/FVC (forced vital capacity) ratio<0.7 at Screen Visits 1 and 2; and
(c) increase in FEV₁ of 12%, over the pre-albuterol FEV₁ within 30 min after inhalation of up to 4 puffs of albuterol delivered by Metered Dose Inhaler (MDI) with spacer at the Screen Visit 1.

Male patients with stable, moderate-to-severe chronic obstructive pulmonary disease are administered MN-221 in the form of a tablet or a capsule, preferably administered once daily, though less frequent, such as biweekly or once weekly administrations, and more frequent, such as 2-3 time daily administrations, are also contemplated. Mean changes in one or more of FEV1, FEV1 (% predicted), PEFR, PEFR (% predicted), and PEF are measured to demonstrate the efficacy of orally administered MN-221 for treating male patients.

What is claimed is:

1. A method of treating a human male patient suffering from a medical condition that is responsive to bedoradrine or a pharmaceutically acceptable salt thereof comprising enterally administering an effective amount of bedoradrine or a pharmaceutically acceptable salt thereof to a human male patient in need thereof.

2. The method of claim 1 in which the effective amount of bedoradrine or a pharmaceutically acceptable salt thereof is administered orally.

3. The method of claim 1 in which the effective amount of bedoradrine or a pharmaceutically acceptable salt thereof is administered via a suppository.

4. The method of claim 1 in which the effective amount of bedoradrine or a pharmaceutically acceptable salt thereof is administered rectally as an enema.

5. The method of claim 1, in which the effective amount of bedoradrine or a pharmaceutically acceptable salt thereof ranges from about 0.3 mg to about 50 mg per day, about 0.3 mg to about 40 mg per day, about 1.5 mg to about 25 mg per day, about 3 mg to about 15 mg per day, or about 5 mg to about 10 mg per day.

6. The method of claim 1, in which the effective amount of bedoradrine or a pharmaceutically acceptable salt thereof is about 40 mg/day, or ranges from about 30 mg to about 50 mg per day.

7. The method of claim 1 in which the effective amount of bedoradrine or a pharmaceutically acceptable salt thereof ranges from about 0.3 mg to about 15 mg per day.

8. The method of claim 1 in which an effective amount of bedoradrine or a pharmaceutically acceptable salt thereof administered enterally ranges from about 0.5 mg to about 5 mg per day.

9. The method of claim 1 in which the medical condition comprises an obstructive airways disease.

10. The method of claim 1 in which the medical condition is chronic obstructive pulmonary disease (COPD).

11. The method of claim 1 in which the medical condition is acute exacerbation of COPD.

12. The method of claim 1 in which the medical condition is chronic obstructive pulmonary disease requiring hospitalization.

13. The method of claim 10 in which the human male patient exhibits improved respiratory function.

14. The method of claim 13 in which the improved respiratory function is characterized by improvement in one or more of dyspnea, respiratory rate, wheezing, accessory muscle use, supplemental oxygen use, or a composite pulmonary function score comprised of two or more of the foregoing measures.

15. A method of treating a human male patient suffering from chronic obstructive pulmonary disease (COPD) or an exacerbation thereof comprising orally administering an effective amount of bedoradrine or a pharmaceutically acceptable salt thereof to the human male patient in need thereof.

16. A method of decreasing a length of a hospital stay of a human male patient suffering from acute or exacerbated chronic obstructive pulmonary disease (acute COPD or COPD exacerbation) comprising orally administering an effective amount of bedoradrine or a pharmaceutically acceptable salt thereof to a human male patient suffering from an acute COPD or COPD exacerbation who has checked into or been admitted to a hospital.

17. A method of improving one or more of FEV₁, FEV₁%, FEV₁ (% predicted), PEF, PEFR, or PEFR (% predicted) in a human male patient suffering from an airway constriction or obstructive airways disease comprising orally administering an effective amount of bedoradrine or a pharmaceutically acceptable salt thereof to a human male patient suffering from an airway constriction or obstructive airways disease.

18. The method of claim 17, in which the effective amount of bedoradrine or a pharmaceutically acceptable salt thereof ranges from about 0.3 mg to about 50 mg per day, about 0.3 mg to about 40 mg per day, about 0.3 mg to about 15 mg per day, about 0.5 mg to about 5 mg per day, about 1.5 mg to about 25 mg per day, 3 mg to about 15 mg per day, or about 5 mg to about 10 mg per day.

19. The method of claim 18, in which effective amount of bedoradrine or a pharmaceutically acceptable salt thereof is about 40 mg/day, or ranges from about 30 mg to about 50 mg per day.

20. The method of claim 18, in which the medical condition comprises an obstructive airways disease.

21. The method of claim 18, in which the medical condition is chronic obstructive pulmonary disease (COPD).

22. The method of claim 18 in which the medical condition is acute exacerbation of COPD.

23. The method of claim 18 in which the medical condition is chronic obstructive pulmonary disease requiring hospitalization.

24. A method of reducing the administration of one or more concomitant medications to a human male patient hospitalized for COPD comprising orally administering an effective amount of bedoradrine or a pharmaceutically acceptable salt thereof to a human male patient hospitalized for COPD.

25. The method of claim 24 in which the one or more concomitant medications is selected from the group consisting of β-agonists other than bedoradrine, antimuscarinics and corticosteroids.

26. The method of claim 24 in which the one or more concomitant medications is selected from the group consisting of albuterol, ipratropium, prednisone and prednisolone.
